(19) United States
(12) Patent Application Publication
(10) Pub. No.: US 2015/0057516 A1
(43) Pub. Date: Feb. 26, 2015

(54) FULLY WIRELESS CONTINUOUSLY WEARABLE SENSOR FOR MEASUREMENT OF EYE FLUID

(71) Applicant: CALIFORNIA INSTITUTE OF TECHNOLOGY, PASADENA, CA (US)

(72) Inventors: Muhammad MUJEEB-U-RAHMAN, PASADENA, CA (US); Axel SCHERER, BARNARD, VT (US)

(21) Appl. No.: 14/465,777

(22) Filed: Aug. 21, 2014

Related U.S. Application Data
(60) Provisional application No. 61/869,540, filed on Aug. 23, 2013.

(51) Int. Cl. A61B 3/10; A61B 5/00; H05K 3/00; H05K 3/14; H05K 3/12; A61B 5/145; C23C 16/06

(52) U.S. Cl. CPC A61B 3/101; A61B 5/14517; A61B 5/0022; A61B 5/6821; C23C 16/06; H05K 3/146; H05K 3/1216; H05K 3/0014
USPC 600/346; 600/345; 600/348; 427/2.12

(57) ABSTRACT

Novel methods and systems for monitoring the health of an eye are disclosed. For example, a resonant circuit may be fabricated on a contact lens and this circuit may be coupled to a second circuit having the same resonant current frequency. A change in a property of the eye fluid contacted by the sensor in the contact lens is communicated to an external device and a remedying action is suggested to the wearer.

### CROSS REFERENCE TO RELATED APPLICATIONS

The present application claims priority to U.S. Provisional Patent Application No. 61/869,540, filed on Aug. 23, 2013, the disclosure of which is incorporated herein by reference in its entirety.

### TECHNICAL FIELD

The present disclosure relates to biological sensing. More particularly, it relates to a fully wireless continuously wearable sensor for measurements of eye fluid.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 illustrates an exemplary circuit.

FIG. 2 illustrates a plot of a resonant frequency and current amplitude.

FIG. 3 illustrates primary and secondary circuits connected through coils.

FIG. 4 illustrates an exemplary contact lens with metal traces.

FIG. 5 illustrates an exemplary contact lens with metal traces and specific polymers.

FIG. 6 illustrates differently shaped capacitors.

FIG. 7 illustrates three dimensional capacitors.

FIG. 8 illustrates a three dimensional metal trace.

FIG. 9 illustrates a sensing system.

### SUMMARY

In a first aspect of the disclosure, a resonant sensor is described, the resonant sensor comprising: a first resonant circuit, comprising at least one first resistor, at least one first capacitor and at least one first inductor, wherein the first resonant circuit is configured so that an eye fluid in contact with the resonant sensor will electrically connect at least two points in the first resonant circuit, and a change in at least one property of the eye fluid will effect a change in a resonant behavior of the first resonant circuit.

### DETAILED DESCRIPTION

The human eye is a very sensitive organ and its proper operation depends upon many factors; presence of proper eye fluids (e.g. tear fluid) is a very important one of these factors. Decrease in these fluids leads to a condition commonly known as dry eye. This can lead to discomfort, mild to severe damage to the ocular surface and to long term inflammation. Measurement of the eye fluid involves measuring its level as well as its constituents; both of which affect the proper function of the eye. In the present application, a fully wireless and wearable eye sensor is described which can measure the required properties of eye fluids through electrical measurements. The sensor uses resonantly coupled devices, where a change in the resonance properties of the wearable device is induced by a change in the eye fluid. This change in resonance properties is coupled to an external module which relays the information to a data display device to transfer the information to a user or a medical practitioner.

Measurement of eye fluids can provide a very useful measure on the health of the eye. However, most of these measurements are taken at discrete intervals using special instruments, which make it less useful for a person who is at permanent risk of eye damage due to dry eye disease or some other eye disease. Measurement of eye fluids can also be used to measure the concentration of toxins or pollutants in the environment as they become soluble in the eye fluids. Development of minimally invasive and continuous sensors for this purpose can lead to many advantages for such applications.

In the present application, the design of sensors is described utilizing electrical resonators. This type of sensor can operate in both active and passive modes. The design is very flexible and can be targeted to detect different chemical species in the eye fluid.

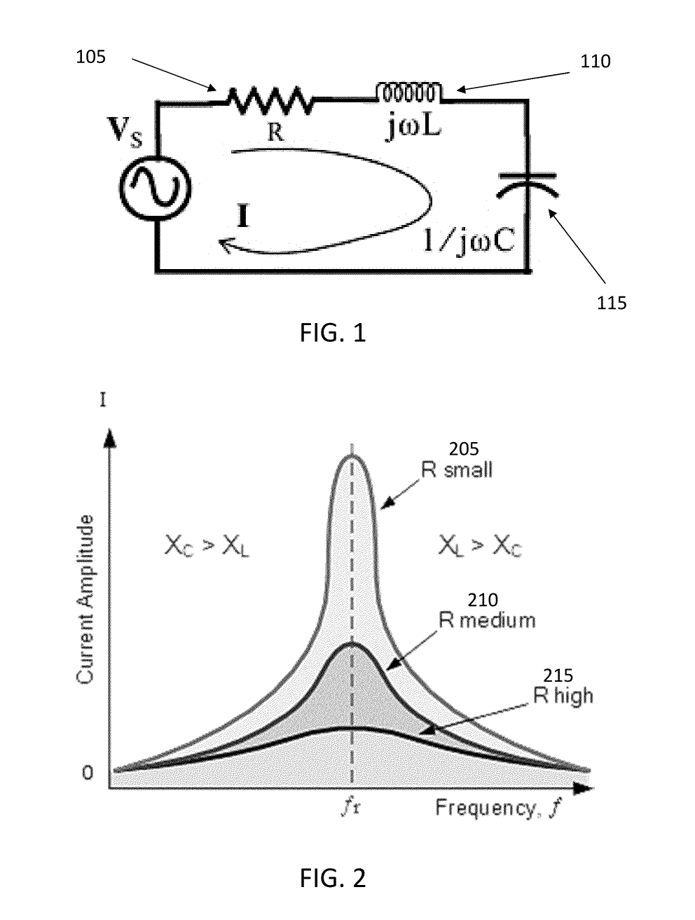

As known to the person skilled in the art, a passive electrical resonator consists of two different energy storing elements, i.e. an inductor and a capacitor, and an energy dissipating resistor, due to the finite (non zero) conductivities of materials used. These electrical elements can be connected in series or parallel. A series circuit is shown as an example in FIG. 1. In FIG. 1, a resistor (105), a capacitor (115) and an inductor (110) are visible.

The current amplitude in the circuit of FIG. 1 depends on the frequency of the electrical current flowing through the circuit, due to the frequency dependence of capacitive and inductive impedances. A typical response can be plotted as in FIG. 2. As known to the person skilled in the art, an alternative current (AC), that is a current that is not constant, but has a value varying in time, interacts in a fundamentally different way with discrete components of an electrical circuit. The reason is that the capacitance and the inductance, in particular, have a value that is a function of the frequency of the current. For example, a current may have a sinusoidal temporal variation.

In FIG. 2, three exemplary cases are displayed, with a low resistance R (205), a medium resistance (210) and a high resistance (215). As known to the person skilled in the art, a low resistance will give a high resonance peak for the current, while a high resistance with give a low resonance peak value for the current in the circuit. Therefore, for example, a change in the value of the resistance can be detected by its effect on the amplitude of the resonant current in the circuit. Since this change in value can be high due to resonant effect, then the circuit is able to detect small changes in resistance at or close to the resonant frequency.

In circuits similar to that of FIG. 2, the frequency for the maximum (e.g. peak) current amplitude corresponds to a resonance conditions given by:

$$fr = \frac{1}{2 π √ LC} ( Hz )$$

where fᵣ is the resonant frequency, L is the inductance and C is the capacitance. The frequency is measured in Hz. The height and sharpness of the peak at this resonant frequency fᵣ is given by another quantity described as the quality factor Q:

$$Q = \frac{1}{R} √ \frac{L}{C}$$

where R is the resistance, L is the inductance and C is the capacitance.

According to the equations above, the magnitude of the resonant frequency depends upon the value of frequency-dependent parameters, such as capacitive and inductive impedances, C and L respectively. The quality factor Q (and other quantities such as the maximum amplitude of the resonant current) depends upon the energy dissipating element as well, such as the resistance R.

If an external signal loads the circuit, for example by providing a resistance path for the current, the external load changes the resonance behavior based upon the type of loading. A change in L or C will change the resonant frequency and a change in R will change the height of the resonant peak and its width.

In some embodiments, the sensors of the present disclosure comprise two coils which connect two circuits. The coils can be termed primary and secondary. The primary and secondary coils can connect two circuits through their induced electrical effects and magnetic fluxes. In some embodiments, a first circuit with one coil can be implanted in a biological tissue or organ, such as the eye. The other circuit with the second coil may be external to the body. Through the electromagnetic effects active between the two coils, the two separate circuits can be coupled.

For example, the secondary coil may be in a circuit embedded in the eye, where changes in the eye fluids may affect the electrical properties of the circuit. Through the secondary coil coupling to the primary coil, where the primary coil is part of a circuit external to the body, the measurement related to eye fluids can be communicated externally to another circuit, and can then be read by a user or medical practitioner.

In the present disclosure, the resonator circuits can be designed to be loaded by the sensing signal in different ways. For example, two electrodes of the resonator circuit may be open to direct contact by the eye fluid, while the remaining components of the circuit are electrically insulated from the eye fluid. The eye fluid, in this embodiment, will act as a conductive channel between the two electrodes. The conductivity of the eye fluid depends on various factors, such as the concentration of chemical and biological components, and the amount of water. For example, if the eye is dry, less eye fluid will be present; therefore its conductivity will decrease. In this case, the resistance between the two electrodes would increase, thereby changing the resonance frequency of the resonator circuit. In one embodiment, a change in the eye fluids conductivity will change the value of the resistance between two separate coils. The coils can act as inductors.

In another embodiment, a change in the eye fluid's conductivity will change the series resistance within one coil. Therefore, different embodiments may have different numbers of coils. As known to the person skilled in the art, coils may have different amounts of windings or turns, which may affect the total inductance of the coils.

In yet another embodiment, a change in the capacitance between the coil turns or between different coils is caused by a change in the permittivity of the eye fluid. As known to the person skilled in the art, the permittivity is a property that can affect the value of electrical quantities. All these changes, as listed in the different embodiments above, can affect the resonance behavior of the secondary coil, which affects the primary coil due to resonant coupling between the two, as known to the person skilled in the art.

The change in the resonance of the implanted coil (‘secondary’) affects another coil (‘primary’), which is in resonance with the primary coil. The primary and secondary coils may have the same resonance frequency. The primary coil is outside the body. This change is detected as a change in the voltage across the primary inductor by a voltage reader and is indicative of the corresponding measurement of eye fluids.

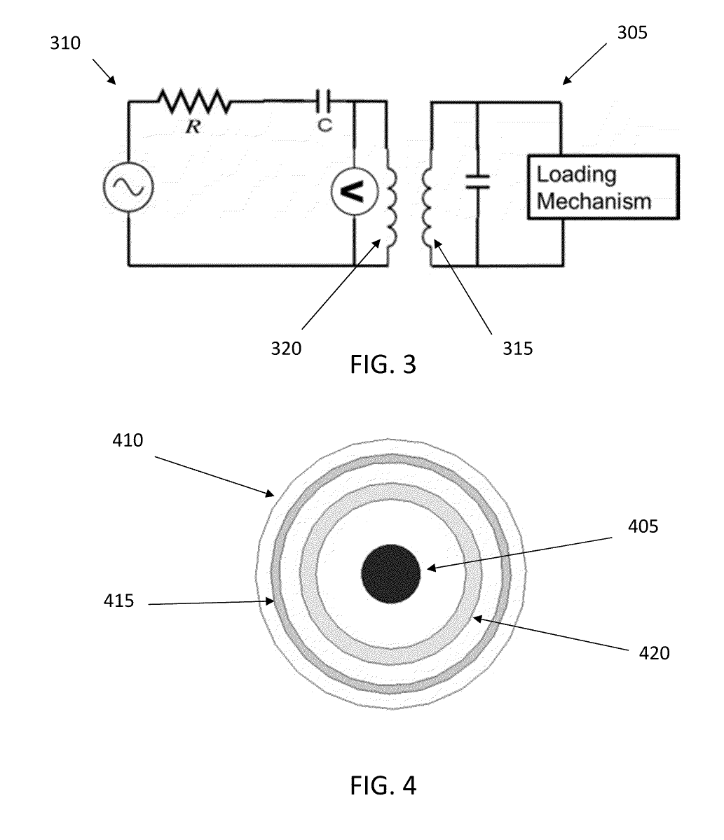

In FIG. 3, the secondary circuit is illustrated as a parallel circuit, but in other embodiments series circuits may be used. In FIG. 3, a secondary circuit (305) is shown, which can be implanted in the human eye. A primary circuit (310) is external and is used to retrieve electrical measurements from the secondary circuit (305). The primary coil (320) couples to the secondary coil (315). In other words, the primary circuit (310) can be part of an external device, while the secondary circuit (305) can be part of the device in the contact lens. The two primary (320) and secondary (305) circuits can be coupled through their respective coils.

The overall sensors of the present disclosure can be designed as a combination of electrical elements in the form of distributed inductors, capacitors and resistors made with noble metals on a wearable contact lens. This is a minimally invasive design and it is user-replaceable. In this embodiment, the sensor is a contact lens.

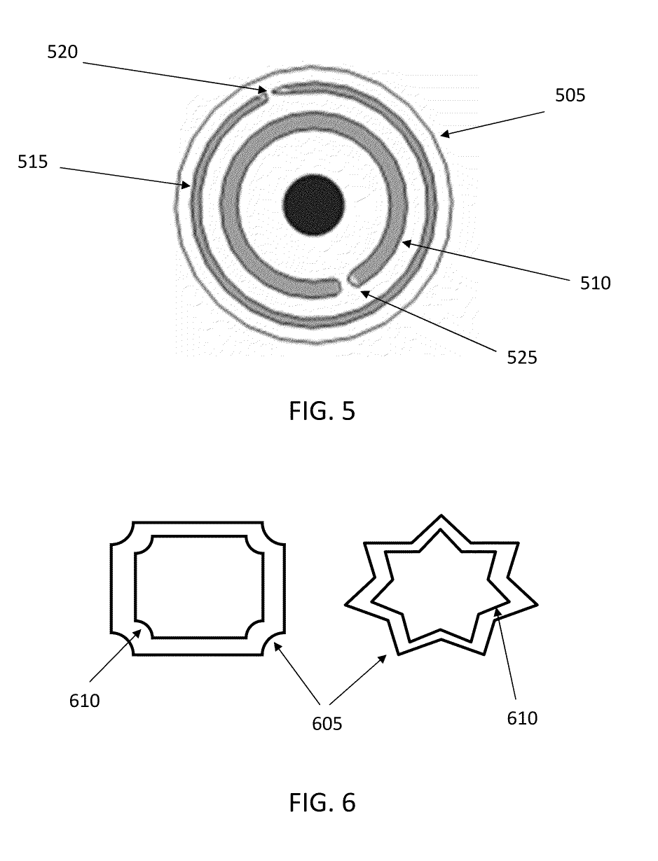

The sensor is on the eye side so that it can access eye fluids more easily. The substrate can be a biocompatible soft material which can support the fabrication process as well as the sensor operation inside the eye. Some example designs are shown in FIGS. 4-5.

In FIG. 4, a contact lens (410) is illustrated, where the pupil is visible schematically (405). Exemplary metal traces (415, 420) are also illustrated. These traces may be optionally covered with polymer to avoid metal-eye contact. The polymer can be water permeable to allow eye fluid access the sensor.

In FIG. 5 another example of a contact lens sensor (505) is illustrated. Metal traces (510, 515) are visible and optionally some parts of the metal traces (510, 515), specifically parts (520, 525), may be covered with selective polymers or hydrogels.

The exposed edges (520, 525) can be coated with different coatings, which are sensitive to different biological species and this converts this basic system into a sensor for specific targets. For example, a coating sensitive to a particular biological entity may be used, and the sensor will detect the presence or absence of that particular biological entity.

The value of the inductance and capacitance in the resonant circuit can be controlled to make the device resonate at a desired frequency where electromagnetic absorption through the substrate and the eye is minimal or where the electromagnetic band is most suitable in terms of signal quality. External inductors and/or capacitors can be added to achieve the desired frequency range, if needed.

The capacitance can be controlled by controlling the shape and material of the electrical elements, and by increasing the cross sectional overlap. One example is to use twisted conductor lines to increase the cross-sectional area of the capacitor as shown in FIG. 6. In FIG. 6, a first conductor (605) constitutes one plate of a capacitor, while a second conductor (610) constitutes a second plate. Different shapes will give a different capacitance. Interdigitated structures can also be used to increase capacitance.

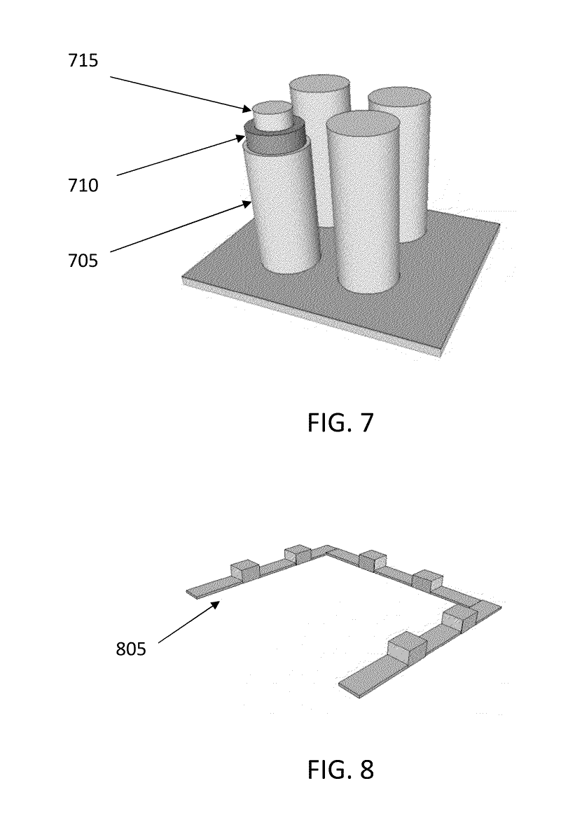

The capacitance can also be controlled or increased by increasing the surface area of the capacitor using micromachining and producing 3D capacitors. This is shown in FIG. 7.

In FIG. 7, a first conductor (705) is separated from a second conductor (715) by a dielectric layer (710).

The inductance can be controlled or increased by using raised structures as shown in the 3D geometry, for example, of FIG. 8, using micromachining. In FIG. 8, a metal track (805) is shown, which can be used to form some or all of the electrical components of a circuit.

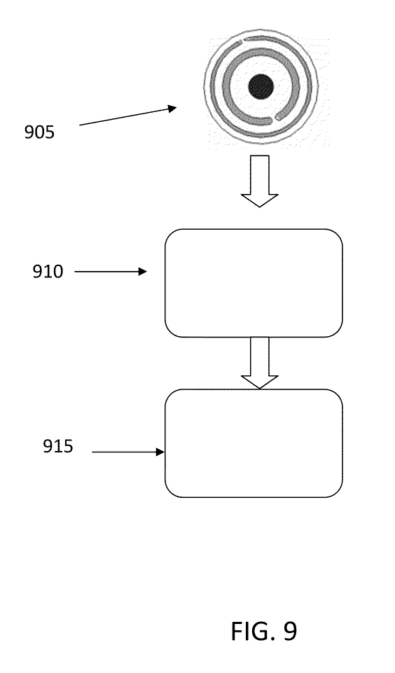

Therefore, as described in the present disclosure, a wearable contact lens comprises a suitable sensor on it. The sensor part can face the eye. The sensor will be resonantly coupled to an external device which can be kept in closed vicinity of the eye(s) (e.g. on glasses). This external device can then connect (e.g. remotely) to a smartphone or other computers, for continuous data storage, showing data trends and displaying proper messages and signal levels. This system is shown in FIG. 9.

In the system illustrated in FIG. 9, a contact lens with a sensor is illustrated (905). The lens (905) is connected (e.g. coupled) to an external device (910). The external device (910) may have a resonator coupled to the implanted resonator, through the primary and secondary coils as described above in the present disclosure. The device (910) may also comprise a power supply and a communication device, which allows communication (e.g. remote) with a computer, tablet or smartphone (915). The smartphone (915) may have a smart application (App) to display sensor data and actuate corresponding actions (e.g. displaying a message ‘put drops in the left eye’).

In some embodiments, the sensor has metal traces, which form a resonator circuit where the role of the resistance (or variation thereof) is played by the eye fluid. For example, the circuit may have two open electrodes where the resistor should be and the electrodes are configured so as to allow the eye fluid to be in contact with the electrodes. Therefore, the eye fluid, being an electrically conducting fluid with a resistance, will short the two electrodes and act as a resistance for the resonator circuit. The conductivity of the eye fluid will change depending, for example, on the amount of eye fluid. Therefore, in some embodiments, a dry eye will have a different resistance than a lubricated eye. In such cases, the resonant circuit will detect the change in resistance through the change in the resonant frequency.

In some embodiments, chemical or biological agents present in the atmosphere can be detected by eye sensors such as the sensors described in the present disclosure, which are embedded in a contact lens. When an agent is present in the air, for example Anthrax, the agent will most likely interact with the surface of the eyes as it is normally exposed and more permeable than skin. In this way, a sensor can detect the presence of such agent by integrating a corresponding chemical sensor in the contact lens.

In some embodiments, the resistor of a circuit may be the parasitic resistance of the inductance and/or the capacitance in the circuit, instead of a discrete resistor. Alternatively, the resistor may be the eye fluid connected between two electrodes of the circuit, as described above in the present disclosure. In some embodiments, all these different types of resistance may be coexistent and indicated overall with the term ‘resistor’.

Different methods may be used to fabricate the devices of the present disclosure.

A suitable metal (e.g. gold) plated printed circuit board (PCB) on flexible biocompatible substrate (e.g. polyimide) may be used. Direct metal coating on flexible polymers (e.g. PET) using thin film deposition methods (e.g. physical vapour deposition PVD, or chemical vapour deposition CVD) or thick film methods (e.g. screen printing) may be used.

The flexible substrates can have the shape of a contact lens to begin with or can be shaped after fabrication using thermal processing (e.g. hot embossing or vacuum forming).

A hole can be made in the contact lens in the pupil area if more transparency is needed. This can be useful for PCB based systems.

The sensors can be used on the skin as well for simple analysis of level of sweating or conductivity of sweat itself. Multiple such sensors can be used to determine the required information.

Functionalization can be done by dip-coating the sensitive area (the exposed area) of the sensor with a hydrogel or a polymer containing the desired chemistry (e.g. ion-sensitive membranes). The mixtures can also be pipetted on the desired area and let to gel or dry in-situ.

In some embodiments, the sensors can be fabricated on contact lenses (or any material already shaped for final application) using stencils (e.g. stick-n-peel type).

Aligned lithography can be done for multi-step fabrication (e.g. passivating parts of metal lines).

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art as a complete disclosure and description of how to make and use the embodiments of the disclosure, and are not intended to limit the scope of what the inventor/inventors regard as their disclosure.

Modifications of the above-described modes for carrying out the methods and systems herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

What is claimed is:

1. A resonant sensor comprising:
a first resonant circuit, comprising at least one first resistor, at least one first capacitor and at least one first inductor, wherein the first resonant circuit is configured so that an eye fluid in contact with the resonant sensor will electrically connect at least two points in the first resonant circuit, and a change in at least one property of the eye fluid will effect a change in a resonant behavior of the first resonant circuit.

2. The resonant sensor of claim 1, wherein a shape of the at least one first capacitor, of the at least one first resistor, and/or the at least one first inductor is configured so as to give a desired value of a resonant current frequency.

3. The resonant sensor of claim 1, wherein the at least one first resistor, at least one first capacitor and at least one first inductor are metal traces on a wearable contact lens.

4. The resonant sensor of claim 3, wherein a shape, length and overlap of the metal traces is configured so as to give a desired value for a resonant current frequency of the first resonant circuit.

5. A system comprising the resonant sensor of claim 1, further comprising:
a second resonant circuit, comprising at least one second resistor, at least one second capacitor and at least one second inductor, wherein the at least one second inductor is configured to be electromagnetically coupled to the at least one first inductor, and wherein the first and second resonant circuits have a same resonant current frequency; and
a reader configured to communicate with the second resonant circuit, thereby obtaining a measurement of the change in the resonant current frequency.

6. A system comprising the resonant sensor of claim 4, further comprising:
a second resonant circuit, comprising at least one second resistor, at least one second capacitor and at least one second inductor, wherein the at least one second inductor is configured to be electromagnetically coupled to the at least one first inductor, and wherein the first and second resonant circuits have a same resonant current frequency; and
a reader configured to communicate with the second resonant circuit, thereby obtaining a measurement of the change in the resonant frequency.

7. The resonant sensor of claim 1, wherein the at least one property of an eye fluid is its resistance or conductance.

8. The resonant sensor of claim 1, wherein the at least one property of an eye fluid is a concentration of a biological or chemical agent.

9. The resonant sensor of claim 8, wherein the biological or chemical agent is Anthrax.

10. A method to monitor a health state of an eye, the method comprising:
applying the system of claim 6 to the eye;
measuring the at least one property of the eye fluid;
detecting the change in the resonant current frequency;
relating the change in the resonant frequency to the change in the at least one property of the eye fluid; and
communicating the change in the at least one property of the eye fluid.

11. The method of claim 10, further comprising, based on the communicating, and suggesting a remedying action to a wearer of the wearable contact lens.

12. A method to fabricate the resonant sensor of claim 1, comprising printing a metallic circuit on a flexible biocompatible substrate.

13. A method to fabricate the resonant sensor of claim 1, comprising direct metal coating on a flexible polymer by thin film deposition or thick film deposition.

14. The method of claim 13, wherein the thin film deposition is physical vapor deposition or chemical vapor deposition.

15. The method of claim 13, wherein the thick film deposition is screen printing.

16. The resonant sensor of claim 1, wherein the first resonant circuit is fabricated on a flexible substrate shaped like a contact lens.

17. The method of claim 12, further comprising shaping the flexible biocompatible substrate like a contact lens by thermal processing.

18. The method of claim 17, wherein the thermal processing is hot embossing or vacuum forming.

19. The resonant sensor of claim 16, further comprising a hole in a pupil area to increase transparency.

20. A method to measure sweat on a skin with the resonant sensor of claim 1, comprising configuring the resonant sensor to detect at least one property of sweat.

21. The method of claim 12, further comprising functionalizing the resonant sensor with a hydrogel or a polymer containing a desired chemistry.

22. The method of claim 21, wherein the polymer is an ion-sensitive membrane.
